(19) United States
(12) Patent Application Publication
(10) Pub. No.: US 2024/0034747 A1
(43) Pub. Date: Feb. 1, 2024

(54) RECRYSTALLIZATION METHOD FOR NMN PREPARATION

(71) Applicant: QINGDAO RESTORE BIOTECHNOLOGY CO., LTD., Qingdao (CN)

(72) Inventors: Mengmeng Chen, Qingdao (CN); Bingqiang Zhang, Qingdao (CN); Yu Zhang, Qingdao (CN); Yi Zhao, Qingdao (CN); Yuan Zhang, Qingdao (CN); Gaofeng Zhang, Qingdao (CN); Xueqi Fu, Qingdao (CN); Wei Zou, Qingdao (CN)

(21) Appl. No.: 18/377,942

(22) Filed: Oct. 9, 2023

(30) Foreign Application Priority Data
Aug. 7, 2023 (CN) 202310983672.8

(51) Int. Cl. C07H 19/048; C07H 1/06; C12P 19/30

(52) U.S. Cl. CPC C07H 19/048; C07H 1/06; C12P 19/30

(57) ABSTRACT

The present invention belongs to the technical field of NMN preparation, and particularly relates to a recrystallization method for NMN preparation, comprising the following steps: step 1. preparation of raw materials: preparing nicotinamide, nucleic acid, ribose-5-phosphate and phosphoric acid; step 2. preparation of reaction solution: preparing a suitable buffer, dissolving nicotinamide, ribose, ribose-5-phosphate in the buffer, and adjusting the concentration of the solution; step 3. activated phosphorylation reaction: adding phosphoric acid to the buffer of the dissolved nicotinamide and ribose, and stirring the mixture at appropriate temperature and pH conditions to make reactions take place in the mixture; step 4. addition of enzyme for catalysis; step 5. separation and purification: separating and purifying the reacted mixture to remove unreacted substrates and synthetase. The present invention is convenient for improving the crystal purity of NMN and improving the crystal quality, and is convenient for users to prepare NMN crystals.

### TECHNICAL FIELD

The present invention belongs to the technical field of NMN preparation, and particularly relates to a recrystallization method for NMN preparation.

### BACKGROUND

NMN (β-nicotinamide mononucleotide) is a naturally occurring bioactive nucleotide. Since nicotinamide belongs to vitamin B3, NMN belongs to the category of vitamin B derivatives. NMN is widely involved in many biochemical reactions in the human body and is closely related to immunity and metabolism. In the human body, NMN is the most direct precursor of NAD+, and the function thereof is reflected by NAD+. NAD+ is also known as coenzyme I, and the full name is nicotinamide adenine dinucleotide, which is present in every cell and involved in thousands of reactions. Nicotinamide adenine dinucleotide (NAD) molecules play an important role in various cellular metabolic reactions, and provide an important support for cells to maintain vitality. The decline in NAD during aging is believed to be a major cause for diseases and disability such as hearing and vision loss, cognitive and motor dysfunction, immunodeficiency, and arthritis, dysbolism and cardiovascular diseases resulting from autoimmune inflammatory reaction disorders.

NMN crystals produced by existing NMN preparation methods have low purity, NMN crystals are easy to be doped with impurities, and the NMN preparation methods are relatively simple, which are inconvenient for users to select preparation methods according to needs. Therefore, a recrystallization method for NMN preparation is proposed to solve the above problems.

### SUMMARY

The purpose of the present invention is to provide a recrystallization method for NMN preparation, which can improve the crystal purity of NMN and improve the crystal quality, and is convenient for users to prepare NMN crystals.

The present invention adopts the following specific technical solution:

A recrystallization method for NMN preparation, comprising the following steps:
Step 1. preparation of raw materials: preparing nicotinamide, nucleic acid, ribose-5-phosphate and phosphoric acid;
Step 2. preparation of reaction solution: preparing a suitable buffer, dissolving nicotinamide, ribose, ribose-5-phosphate in the buffer, and adjusting the concentration of the solution;
Step 3. activated phosphorylation reaction: adding phosphoric acid to the buffer of the dissolved nicotinamide and ribose, and stirring the mixture at appropriate temperature and pH conditions to make reactions take place in the mixture;
Step 4. addition of enzyme for catalysis: adding NMN synthetase as a catalyst, wherein the synthetase can promote the reaction of nicotinamide and phosphoric acid to generate NMN, and adjusting the amount of the synthetase added, the reaction time and the temperature according to experimental conditions;
Step 5. separation and purification: separating and purifying the reacted mixture to remove unreacted substrates and synthetase;
Chromatography: using various chromatographic techniques to separate NMN from the mixture;
Dialysis: separating the small molecules of NMN from the large molecules of substrates and synthetase by osmosis of the solution;
Step 6. drying: drying the purified NMN solution to remove residual solvent and water, wherein the drying method is concentration and freeze-drying;
Step 7. dissolving: adding an appropriate amount of solvent to the NMN solution to completely dissolve the NMN;
Step 8. filtrate treatment: using a microporous filter membrane or filter paper to filter the dissolved NMN solution, thus to remove suspended solids and impurities;
Step 9. selection of crystallization solvent: selecting an appropriate crystallization solvent, wherein commonly used solvents include ethanol and acetone;
Step 10. concentration: putting the filtrate into an appropriate container, and removing the solvent from the NMN solution by heating, ventilation or decompression to concentrate the solution;
Step 11. preparation of crystal seeds and crystallization: preparing a small batch of crystallized NMN as crystal seeds, obtaining the crystal seeds by standing the solution at a low temperature or adding a small amount of crystallized NMN crystals, adding the crystal seeds prepared in advance gradually into the concentrated NMN solution, stirring well, stirring the solution continuously and controlling the temperature after the crystal seeds are added so as to promote the formation of crystals, adjusting the stirring speed and temperature to make the crystallization proceed slowly so as to obtain larger and purer crystals, using an optical microscope or other appropriate methods to observe characteristics of the crystals such as morphology and particle size, and making a record;
Step 12. recrystallization collection and drying: using filter paper, fiberglass filter membrane or other suitable filtration devices to separate the crystals from the solution, wherein the recrystallization separation method is template crystallization method, aeration crystallization method, cryogenic crystallization method or gradual cooling method, washing the separated crystals with a cold solvent to remove impurities attached to the surface, placing the washed crystals in a dryer or fume hood to dry the crystals fully, and packaging and storing the dried crystals.

In a preferred solution, the buffer is phosphate buffer.

In a preferred solution, the reaction temperature of the activated phosphorylation reaction is 25-37° C., and the reaction PH value of the activated phosphorylation reaction is 7.0-8.5.

In a preferred solution, the addition ratio of the NMN synthetase to nicotinamide is 1:20, the reaction temperature of the NMN synthetase is 30-37° C., the reaction PH value of the NMN synthetase is 7.5-8.5, and the reaction time of the NMN synthetase is 1-4 hours.

In a preferred solution, the chromatographic techniques are column chromatography and liquid chromatography.

In a preferred solution, the NMN dissolving solvent is deionized water.

In a preferred solution, the template crystallization method comprises: adding a suitable crystallization template, such as tiny crystals or other kinds of crystals, into the NMN solution to guide the generation of new crystals and improve the yield and purity, and adjusting the stirring speed, temperature and pH value of the solution to promote the formation of crystals.

In a preferred solution, the aeration crystallization method comprises: stirring the NMN solution and making a gas flow into, such as blowing nitrogen or air into the solution to change the solubility and physical conditions of the solution, thus to promote the formation of crystals.

In a preferred solution, the cryogenic crystallization method comprises: placing the NMN solution in a low temperature environment, such as using a low temperature ice bath or liquid nitrogen refrigeration, to increase the crystallization rate and improve the crystal quality.

In a preferred solution, the gradual cooling method comprises: controlling the temperature of the NMN solution to gradually reduce the temperature, thus to promote the control of crystal growth and crystal size.

The present invention has the following technical effects: improving purity: recrystallization is an effective method, through which unreacted substrates, impurities and impure substances can be removed, so that the purity of a product can be increased;
Improving yield: through a recrystallization process, the formation of crystals can be promoted, and the crystallization conditions can be controlled, so that the generation rate of crystals is increased, and the yield of the product is improved;
Improving crystal purity: through the recrystallization process, the crystallization rate and crystal growth environment can be controlled, so that the crystal shape is more regular, the crystal size is more uniform, and the crystal quality is improved; Removing impurity: through the recrystallization method, the attached impurities on crystal surface can be removed by the steps such as washing, so that the final product can be purer;
Convenient storage and packaging: after recrystallization, the NMN crystals has a regular shape, is adequately dried, and is convenient for storage and packaging, and the shelf life of the product is extended;
A variety of different crystallization methods provide users with different crystallization selections, which is convenient for users to select appropriate crystallization methods according to respective production conditions.

### DESCRIPTION OF DRAWINGS

FIG. 1 is an operation flow chart of a recrystallization method for NMN preparation provided in an embodiment of the present invention.

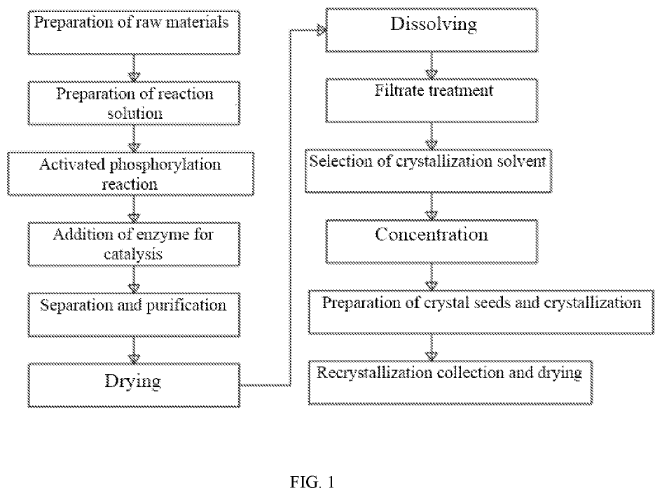

### DETAILED DESCRIPTION

To make the above-mentioned purpose, features and advantages of the present invention more clear and understandable, specific embodiments of the present invention will be described below in detail in combination with the drawings of the description.

Many details are elaborated in the following description for convenience of fully understanding the present invention. However, the present invention can also be implemented in other modes different from those described herein. Those skilled in the art may make similar promotion without departing from the connotation of the present invention. Therefore, the present invention is not limited by specific embodiments disclosed below.

Secondly, the term “an embodiment” or “embodiment” herein means a specific feature, structure or characteristic that may be included in at least one implementation of the present invention. “In a preferred embodiment” appearing in different places in the description does not mean the same embodiment, nor a separate or selective embodiment that is mutually exclusive with other embodiments.

Thirdly, the present invention is described in detail in combination with drawings. When elaborating embodiments of the present invention, for ease of presentation, a sectional view representing a device structure will not be locally enlarged according to an ordinary scale, and the drawings are only examples, which shall not be regarded as limiting the protection scope of the present invention. In addition, the three-dimensional space dimensions of length, width and depth shall be included in actual production.

### Embodiment 1

A recrystallization method for NMN preparation, comprising the following steps:
Step 1. preparation of raw materials: preparing nicotinamide, nucleic acid, ribose-5-phosphate and phosphoric acid;
Step 2. preparation of reaction solution: preparing a suitable buffer, dissolving nicotinamide, ribose, ribose-5-phosphate in the buffer, and adjusting the concentration of the solution;
Step 3. activated phosphorylation reaction: adding phosphoric acid to the buffer of the dissolved nicotinamide and ribose, and stirring the mixture at appropriate temperature and pH conditions to make reactions take place in the mixture;
Step 4. addition of enzyme for catalysis: adding NMN synthetase as a catalyst, wherein the synthetase can promote the reaction of nicotinamide and phosphoric acid to generate NMN, and adjusting the amount of the synthetase added, the reaction time and the temperature according to experimental conditions;
Step 5. separation and purification: separating and purifying the reacted mixture to remove unreacted substrates and synthetase;
Chromatography: using various chromatographic techniques to separate NMN from the mixture;
Dialysis: separating the small molecules of NMN from the large molecules of substrates and synthetase by osmosis of the solution;
Step 6. drying: drying the purified NMN solution to remove residual solvent and water, wherein the drying method is concentration and freeze-drying;
Step 7. dissolving: adding an appropriate amount of solvent to the NMN solution to completely dissolve the NMN;
Step 8. filtrate treatment: using a microporous filter membrane or filter paper to filter the dissolved NMN solution, thus to remove suspended solids and impurities;
Step 9. selection of crystallization solvent: selecting an appropriate crystallization solvent, wherein commonly used solvents include ethanol and acetone;
Step 10. concentration: putting the filtrate into an appropriate container, and removing the solvent from the NMN solution by heating, ventilation or decompression to concentrate the solution;
Step 11. preparation of crystal seeds and crystallization: preparing a small batch of crystallized NMN as crystal seeds, obtaining the crystal seeds by standing the solution at a low temperature or adding a small amount of crystallized NMN crystals, adding the crystal seeds prepared in advance gradually into the concentrated NMN solution, stirring well, stirring the solution continuously and controlling the temperature after the crystal seeds are added so as to promote the formation of crystals, adjusting the stirring speed and temperature to make the crystallization proceed slowly so as to obtain larger and purer crystals, using an optical microscope or other appropriate methods to observe characteristics of the crystals such as morphology and particle size, and making a record;
Step 12. recrystallization collection and drying: using filter paper, fiberglass filter membrane or other suitable filtration devices to separate the crystals from the solution, wherein the recrystallization separation method is template crystallization method, washing the separated crystals with a cold solvent to remove impurities attached to the surface, placing the washed crystals in a dryer or fume hood to dry the crystals fully, and packaging and storing the dried crystals;

The buffer is phosphate buffer, the reaction temperature of the activated phosphorylation reaction is 25-37° C., the reaction PH value of the activated phosphorylation reaction is 7.0-8.5, the addition ratio of the NMN synthetase to nicotinamide is 1:20, the reaction temperature of the NMN synthetase is 30-37° C., the reaction PH value of the NMN synthetase is 7.5-8.5, the reaction time of the NMN synthetase is 1-4 hours, the chromatographic techniques are column chromatography and liquid chromatography, the NMN dissolving solvent is deionized water, and the template crystallization method comprises: adding a suitable crystallization template, such as tiny crystals or other kinds of crystals, into the NMN solution to guide the generation of new crystals and improve the yield and purity, and adjusting the stirring speed, temperature and pH value of the solution to promote the formation of crystals.

### Embodiment 2

A recrystallization method for NMN preparation, comprising the following steps:
Step 1. preparation of raw materials: preparing nicotinamide, nucleic acid, ribose-5-phosphate and phosphoric acid;
Step 2. preparation of reaction solution: preparing a suitable buffer, dissolving nicotinamide, ribose, ribose-5-phosphate in the buffer, and adjusting the concentration of the solution;
Step 3. activated phosphorylation reaction: adding phosphoric acid to the buffer of the dissolved nicotinamide and ribose, and stirring the mixture at appropriate temperature and pH conditions to make reactions take place in the mixture;
Step 4. addition of enzyme for catalysis: adding NMN synthetase as a catalyst, wherein the synthetase can promote the reaction of nicotinamide and phosphoric acid to generate NMN, and adjusting the amount of the synthetase added, the reaction time and the temperature according to experimental conditions;
Step 5. separation and purification: separating and purifying the reacted mixture to remove unreacted substrates and synthetase;
Chromatography: using various chromatographic techniques to separate NMN from the mixture;
Dialysis: separating the small molecules of NMN from the large molecules of substrates and synthetase by osmosis of the solution;
Step 6. drying: drying the purified NMN solution to remove residual solvent and water, wherein the drying method is concentration and freeze-drying;
Step 7. dissolving: adding an appropriate amount of solvent to the NMN solution to completely dissolve the NMN;
Step 8. filtrate treatment: using a microporous filter membrane or filter paper to filter the dissolved NMN solution, thus to remove suspended solids and impurities;
Step 9. selection of crystallization solvent: selecting an appropriate crystallization solvent, wherein commonly used solvents include ethanol and acetone;
Step 10. concentration: putting the filtrate into an appropriate container, and removing the solvent from the NMN solution by heating, ventilation or decompression to concentrate the solution;
Step 11. preparation of crystal seeds and crystallization: preparing a small batch of crystallized NMN as crystal seeds, obtaining the crystal seeds by standing the solution at a low temperature or adding a small amount of crystallized NMN crystals, adding the crystal seeds prepared in advance gradually into the concentrated NMN solution, stirring well, stirring the solution continuously and controlling the temperature after the crystal seeds are added so as to promote the formation of crystals, adjusting the stirring speed and temperature to make the crystallization proceed slowly so as to obtain larger and purer crystals, using an optical microscope or other appropriate methods to observe characteristics of the crystals such as morphology and particle size, and making a record;
Step 12. recrystallization collection and drying: using filter paper, fiberglass filter membrane or other suitable filtration devices to separate the crystals from the solution, wherein the recrystallization separation method is aeration crystallization method, washing the separated crystals with a cold solvent to remove impurities attached to the surface, placing the washed crystals in a dryer or fume hood to dry the crystals fully, and packaging and storing the dried crystals;
The buffer is phosphate buffer, the reaction temperature of the activated phosphorylation reaction is 25-37° C., the reaction PH value of the activated phosphorylation reaction is 7.0-8.5, the addition ratio of the NMN synthetase to nicotinamide is 1:20, the reaction temperature of the NMN synthetase is 30-37° C., the reaction PH value of the NMN synthetase is 7.5-8.5, the reaction time of the NMN synthetase is 1-4 hours, the chromatographic techniques are column chromatography and liquid chromatography, the NMN dissolving solvent is deionized water, and the aeration crystallization method comprises: stirring the NMN solution and making a gas flow into, such as blowing nitrogen or air into the solution to change the solubility and physical conditions of the solution, thus to promote the formation of crystals.

### Embodiment 3

A recrystallization method for NMN preparation, comprising the following steps:
Step 1. preparation of raw materials: preparing nicotinamide, nucleic acid, ribose-5-phosphate and phosphoric acid;
Step 2. preparation of reaction solution: preparing a suitable buffer, dissolving nicotinamide, ribose, ribose-5-phosphate in the buffer, and adjusting the concentration of the solution;
Step 3. activated phosphorylation reaction: adding phosphoric acid to the buffer of the dissolved nicotinamide and ribose, and stirring the mixture at appropriate temperature and pH conditions to make reactions take place in the mixture;
Step 4. addition of enzyme for catalysis: adding NMN synthetase as a catalyst, wherein the synthetase can promote the reaction of nicotinamide and phosphoric acid to generate NMN, and adjusting the amount of the synthetase added, the reaction time and the temperature according to experimental conditions;
Step 5. separation and purification: separating and purifying the reacted mixture to remove unreacted substrates and synthetase;
Chromatography: using various chromatographic techniques to separate NMN from the mixture;
Dialysis: separating the small molecules of NMN from the large molecules of substrates and synthetase by osmosis of the solution;
Step 6. drying: drying the purified NMN solution to remove residual solvent and water, wherein the drying method is concentration and freeze-drying;
Step 7. dissolving: adding an appropriate amount of solvent to the NMN solution to completely dissolve the NMN;
Step 8. filtrate treatment: using a microporous filter membrane or filter paper to filter the dissolved NMN solution, thus to remove suspended solids and impurities;
Step 9. selection of crystallization solvent: selecting an appropriate crystallization solvent, wherein commonly used solvents include ethanol and acetone;
Step 10. concentration: putting the filtrate into an appropriate container, and removing the solvent from the NMN solution by heating, ventilation or decompression to concentrate the solution;
Step 11. preparation of crystal seeds and crystallization: preparing a small batch of crystallized NMN as crystal seeds, obtaining the crystal seeds by standing the solution at a low temperature or adding a small amount of crystallized NMN crystals, adding the crystal seeds prepared in advance gradually into the concentrated NMN solution, stirring well, stirring the solution continuously and controlling the temperature after the crystal seeds are added so as to promote the formation of crystals, adjusting the stirring speed and temperature to make the crystallization proceed slowly so as to obtain larger and purer crystals, using an optical microscope or other appropriate methods to observe characteristics of the crystals such as morphology and particle size, and making a record;
Step 12. recrystallization collection and drying: using filter paper, fiberglass filter membrane or other suitable filtration devices to separate the crystals from the solution, wherein the recrystallization separation method is cryogenic crystallization method, washing the separated crystals with a cold solvent to remove impurities attached to the surface, placing the washed crystals in a dryer or fume hood to dry the crystals fully, and packaging and storing the dried crystals; The buffer is phosphate buffer, the reaction temperature of the activated phosphorylation reaction is 25-37° C., the reaction PH value of the activated phosphorylation reaction is 7.0-8.5, the addition ratio of the NMN synthetase to nicotinamide is 1:20, the reaction temperature of the NMN synthetase is 30-37° C., the reaction PH value of the NMN synthetase is 7.5-8.5, the reaction time of the NMN synthetase is 1-4 hours, the chromatographic techniques are column chromatography and liquid chromatography, the NMN dissolving solvent is deionized water, and the cryogenic crystallization method comprises: placing the NMN solution in a low temperature environment, such as using a low temperature ice bath or liquid nitrogen refrigeration, to increase the crystallization rate and improve the crystal quality.

### Embodiment 4

A recrystallization method for NMN preparation, comprising the following steps:
Step 1. preparation of raw materials: preparing nicotinamide, nucleic acid, ribose-5-phosphate and phosphoric acid;
Step 2. preparation of reaction solution: preparing a suitable buffer, dissolving nicotinamide, ribose, ribose-5-phosphate in the buffer, and adjusting the concentration of the solution;
Step 3. activated phosphorylation reaction: adding phosphoric acid to the buffer of the dissolved nicotinamide and ribose, and stirring the mixture at appropriate temperature and pH conditions to make reactions take place in the mixture;
Step 4. addition of enzyme for catalysis: adding NMN synthetase as a catalyst, wherein the synthetase can promote the reaction of nicotinamide and phosphoric acid to generate NMN, and adjusting the amount of the synthetase added, the reaction time and the temperature according to experimental conditions;
Step 5. separation and purification: separating and purifying the reacted mixture to remove unreacted substrates and synthetase;
Chromatography: using various chromatographic techniques to separate NMN from the mixture;
Dialysis: separating the small molecules of NMN from the large molecules of substrates and synthetase by osmosis of the solution;
Step 6. drying: drying the purified NMN solution to remove residual solvent and water, wherein the drying method is concentration and freeze-drying;
Step 7. dissolving: adding an appropriate amount of solvent to the NMN solution to completely dissolve the NMN;
Step 8. filtrate treatment: using a microporous filter membrane or filter paper to filter the dissolved NMN solution, thus to remove suspended solids and impurities;
Step 9. selection of crystallization solvent: selecting an appropriate crystallization solvent, wherein commonly used solvents include ethanol and acetone;
Step 10. concentration: putting the filtrate into an appropriate container, and removing the solvent from the NMN solution by heating, ventilation or decompression to concentrate the solution;
Step 11. preparation of crystal seeds and crystallization: preparing a small batch of crystallized NMN as crystal seeds, obtaining the crystal seeds by standing the solution at a low temperature or adding a small amount of crystallized NMN crystals, adding the crystal seeds prepared in advance gradually into the concentrated NMN solution, stirring well, stirring the solution continuously and controlling the temperature after the crystal seeds are added so as to promote the formation of crystals, adjusting the stirring speed and temperature to make the crystallization proceed slowly so as to obtain larger and purer crystals, using an optical microscope or other appropriate methods to observe characteristics of the crystals such as morphology and particle size, and making a record;
Step 12. recrystallization collection and drying: using filter paper, fiberglass filter membrane or other suitable filtration devices to separate the crystals from the solution, wherein the recrystallization separation method is gradual cooling method, washing the separated crystals with a cold solvent to remove impurities attached to the surface, placing the washed crystals in a dryer or fume hood to dry the crystals fully, and packaging and storing the dried crystals;
The buffer is phosphate buffer, the reaction temperature of the activated phosphorylation reaction is 25-37° C., the reaction PH value of the activated phosphorylation reaction is 7.0-8.5, the addition ratio of the NMN synthetase to nicotinamide is 1:20, the reaction temperature of the NMN synthetase is 30-37° C., the reaction PH value of the NMN synthetase is 7.5-8.5, the reaction time of the NMN synthetase is 1-4 hours, the chromatographic techniques are column chromatography and liquid chromatography, the NMN dissolving solvent is deionized water, and the gradual cooling method comprises: controlling the temperature of the NMN solution to gradually reduce the temperature, thus to promote the control of crystal growth and crystal size.

In the present invention, unreacted substrates, impurities and impure substances can be removed, so that the purity of a product can be increased; through a recrystallization process, the formation of crystals can be promoted, and the crystallization conditions can be controlled, so that the generation rate of crystals is increased, and the yield of the product is improved; through the recrystallization process, the crystallization rate and crystal growth environment can be controlled, so that the crystal shape is more regular, the crystal size is more uniform, and the crystal quality is improved; through the recrystallization method, the attached impurities on crystal surface can be removed by the steps such as washing, so that the final product can be purer; after recrystallization, the NMN crystals has a regular shape, is adequately dried, and is convenient for storage and packaging, and the shelf life of the product is extended.

The above descriptions are only preferred embodiments of the present invention. It should be noted that, for those ordinary skilled in the art, many improvements and polishes may be made without departure from the technical principles of the present invention, and these improvements and polishes should also be deemed to be fallen into the protection scope of the present invention. Structures, devices and operation methods not specifically described and explained in the present invention are implemented in accordance with conventional means in the art unless otherwise specified and limited.

What is claimed is:

1. A recrystallization method for NMN preparation, comprising the following steps:
step 1. preparation of raw materials: preparing nicotinamide, nucleic acid, ribose-5-phosphate and phosphoric acid;
step 2. preparation of reaction solution: preparing a suitable buffer, dissolving nicotinamide, ribose, ribose-5-phosphate in the buffer, and adjusting the concentration of the solution;
step 3. activated phosphorylation reaction: adding phosphoric acid to the buffer of the dissolved nicotinamide and ribose, and stirring the mixture at appropriate temperature and pH conditions to make reactions take place in the mixture;
step 4. addition of enzyme for catalysis: adding NMN synthetase as a catalyst, wherein the synthetase can promote the reaction of nicotinamide and phosphoric acid to generate NMN, and adjusting the amount of the synthetase added, the reaction time and the temperature according to experimental conditions;
step 5. separation and purification: separating and purifying the reacted mixture to remove unreacted substrates and synthetase;
chromatography: using various chromatographic techniques to separate NMN from the mixture;
dialysis: separating the small molecules of NMN from the large molecules of substrates and synthetase by osmosis of the solution;
step 6. drying: drying the purified NMN solution to remove residual solvent and water, wherein the drying method is concentration and freeze-drying;
step 7. dissolving: adding an appropriate amount of solvent to the NMN solution to completely dissolve the NMN;
step 8. filtrate treatment: using a microporous filter membrane or filter paper to filter the dissolved NMN solution, thus to remove suspended solids and impurities;
step 9. selection of crystallization solvent: selecting an appropriate crystallization solvent, wherein commonly used solvents include ethanol and acetone;
step 10. concentration: putting the filtrate into an appropriate container, and removing the solvent from the NMN solution by heating, ventilation or decompression to concentrate the solution;
step 11. preparation of crystal seeds and crystallization: preparing a small batch of crystallized NMN as crystal seeds, obtaining the crystal seeds by standing the solution at a low temperature or adding a small amount of crystallized NMN crystals, adding the crystal seeds prepared in advance gradually into the concentrated NMN solution, stirring well, stirring the solution continuously and controlling the temperature after the crystal seeds are added so as to promote the formation of crystals, adjusting the stirring speed and temperature to make the crystallization proceed slowly so as to obtain larger and purer crystals, using an optical microscope or other appropriate methods to observe characteristics of the crystals such as morphology and particle size, and making a record;
step 12. recrystallization collection and drying: using filter paper, fiberglass filter membrane or other suitable filtration devices to separate the crystals from the solution, wherein the recrystallization separation method is template crystallization method, aeration crystallization method, cryogenic crystallization method or gradual cooling method, washing the separated crystals with a cold solvent to remove impurities attached to the surface, placing the washed crystals in a dryer or fume hood to dry the crystals fully, and packaging and storing the dried crystals.

2. The recrystallization method for NMN preparation according to claim 1, wherein the buffer is phosphate buffer.

3. The recrystallization method for NMN preparation according to claim 1, wherein the reaction temperature of the activated phosphorylation reaction is 25-37° C., and the reaction PH value of the activated phosphorylation reaction is 7.0-8.5.

4. The recrystallization method for NMN preparation according to claim 1, wherein the addition ratio of the NMN synthetase to nicotinamide is 1:20, the reaction temperature of the NMN synthetase is 30-37° C., the reaction PH value of the NMN synthetase is 7.5-8.5, and the reaction time of the NMN synthetase is 1-4 hours.

5. The recrystallization method for NMN preparation according to claim 1, wherein the chromatographic techniques are column chromatography and liquid chromatography.

6. The recrystallization method for NMN preparation according to claim 1, wherein the NMN dissolving solvent is deionized water.

7. The recrystallization method for NMN preparation according to claim 1, wherein the template crystallization method comprises: adding a suitable crystallization template, such as tiny crystals or other kinds of crystals, into the NMN solution to guide the generation of new crystals and improve the yield and purity, and adjusting the stirring speed, temperature and pH value of the solution to promote the formation of crystals.

8. The recrystallization method for NMN preparation according to claim 1, wherein the aeration crystallization method comprises: stirring the NMN solution and making a gas flow into, such as blowing nitrogen or air into the solution to change the solubility and physical conditions of the solution, thus to promote the formation of crystals.

9. The recrystallization method for NMN preparation according to claim 1, wherein the cryogenic crystallization method comprises: placing the NMN solution in a low temperature environment, such as using a low temperature ice bath or liquid nitrogen refrigeration, to increase the crystallization rate and improve the crystal quality.

10. The recrystallization method for NMN preparation according to claim 1, wherein the gradual cooling method comprises: controlling the temperature of the NMN solution to gradually reduce the temperature, thus to promote the control of crystal growth and crystal size.
